(19) United States
(12) Patent Application Publication
(10) Pub. No.: US 2020/0187862 A1
(43) Pub. Date: Jun. 18, 2020

(54) IMPLANTABLE THIN-FILM PROBES

(71) Applicants: Xiangbing JIAO, Tempe, AZ (US); Yuan WANG, Anhui (CN); Quan QING, Chandler, AZ (US)

(72) Inventors: Xiangbing JIAO, Tempe, AZ (US); Yuan WANG, Anhui (CN); Quan QING, Chandler, AZ (US)

(21) Appl. No.: 16/608,603

(22) PCT Filed: Apr. 27, 2018
(86) PCT No.: PCT/US18/29811

§ 371 Date: Oct. 25, 2019

Related U.S. Application Data
(60) Provisional application No. 62/490,960, filed on Apr. 27, 2017.

(51) Int. Cl. A61B 5/00; A61B 5/04

(52) U.S. Cl. CPC A61B 5/6861; A61N 1/0551; A61B 2562/125; A61B 5/04001

(57) ABSTRACT

Implantable thin-films have a variety of uses but due to their geometry and size, they are extremely fragile. The present disclosure presents devices and apparatuses for implantable thin-film probes capable of maintaining their thin-film structure through implantation through the use of degradable sacrificial layers to plant or deposit a thin film structure via a rigid substrate and remove the rigid substrate once the sacrificial layer has dissolved. The disclosed structures and methods are in reference to biocompatible films for use in vivo, but other environments and uses may also benefit from the disclosed structures and techniques, such as prosthetic/assistive devices, chronic deep tissue electrical stimulation, electrical field-directed cell migration for wound healing, nerve/neuron rehabilitation, etc.

### CROSS-REFERENCE TO RELATED APPLICATIONS

This application claims the benefit of U.S. Provisional Application No. 62/490,960, filed Apr. 27, 2017, which is incorporated herein in its entirety.

### STATEMENT OF SUPPORT

The invention was made with U.S. Government support under Grant No. FA9550-16-1-0052 awarded by Air Force Office of Scientific Research and Grant No. R21EB020822 awarded by National Institutes of Health. The U.S. Government has certain rights in the invention.

### BACKGROUND

### 1. Technical Field

The present invention relates generally to implantable thin-films, and more specifically, but not by way of limitation, to biocompatible, implantable thin-film probes implanted via biodegradable sacrificial layers where a substrate maintains the structure of the thin-film until the sacrificial layer dissolves.

### 2. Description of Related Art

Implantable electrodes have been the central unit of implanted sensors and electronics for biomedical research, diagnostics and therapy. Overall, there are two categories of commercialized electrodes that have been used in implantation. First, rigid microscale electrodes are most widely used when accurate positioning into the tissue are required. The major challenge for the chronic use of these probes is the tissue reaction and scar formation around the probe due to the huge mechanical mismatch with the tissue, and micromotions after the implantation, such that the active cells tend to migrate away from the implanted electrodes, which eventually leads to significant degradation of performance. Second, flexible thin-film probes are used where they can conform to the shape of the tissue/organ so that it could bring less irritation to the live cells and better biocompatibility. However, the devices typically can only get in touch with the outmost surface of the tissue, and it is generally difficult to deliver the flexible structure deep below the surface to get closer to the active cells, which reduces the resolution and quality of signals.

Recent advances in nano-bioelectronics and bioresorbable electronics have triggered a new wave of innovations of how to interface artificial devices to live cells and tissues. Several new strategies have been explored to package nanomaterial-enabled devices in flexible forms and to deliver them inside tissue versus just making a surface contact. Specifically, Lieber group developed procedures to implant three-dimensional ultra-flexible macroporous nanoelectronic probes into the brain shortly after freezing the probes in liquid nitrogen so that the rigidity of the probes is temporarily strong enough to penetrate the tissue before thawing in position.¹ More significantly, they have recently demonstrated injectable flexible electronics into tissues, in which packed microporous mesh electronics is directed through a syringe by a flux of liquid, and a high yield of connection can be made using conductive ink printing technique.^{2,3} Nevertheless, these ultra-flexible probes with sub-10 μm size devices which are ideal for long-term implanted applications do not have a well-controlled position or geometry inside the tissue but rather typically spread randomly within a diameter of hundreds of μm up to mm because the soft probes need to be first separated from the fabrication substrate, and then delivered as suspended in medium. On the other hand, Rogers' group proposed transient electronics for implanted application which utilizes biodegradable materials, also known as bioabsorbable materials that are soluble in aqueous solutions or biofluids, specifically for functional components, connection wires and packaging, and the whole circuit will be absorbed by the tissue and disappear after an extended period.⁴ In addition, for the implantation of ultra-thin injectable optoelectronics and sensors, Rogers et al. designed a process to pick up and transfer thin-film devices onto an epoxy-based needle and glue them together with silk-based solution, so that the needle is strong enough to bring the thin-film structure into the tissue and the film can detach soon after the silk glue is dissolved.^{5,6} The precision of insertion is well controlled, whereas the surgical lesion/damage of the tissue is typically wider than 100 μm up to mm in size, possibly because the choice of materials of the supporting structure requires a larger geometry for enough rigidity, and the dissolvable layer is incompatible with lithographic processes which limits scaling down the device size. For minimizing surgery damage, Xie et al. recently demonstrated an ultra-flexible nanoelectronic probe that can be implanted with surgical damage as small as 10 μm.⁷ Nevertheless, such probe is implanted with the aid of an insertion shuttle made of tungsten wire or carbon fiber, which requires an elaborated focus ion beam sharpening process for each shuttle and complicated assembling procedures and therefore cannot be scaled up.

Despite studies demonstrating the importance of matching the size and mechanical properties of the implanted functional devices with live cells and tissues, and the desire to minimize the impact of lesion while maintaining the feasibility of accurate surgery with rigidity, none have solved the task of scaling down the size of probe while maintaining the mechanical strength required for accurate implantation surgery and having the core functional structure small and flexible enough for better interfacing with cells.

### SUMMARY

The present disclosure addresses the challenges known in the art, including a general fabrication framework utilizing an inorganic biodegradable sacrificial layer that can be integrated in a unified top-down lithography procedure for preparing ultra-small probes to accurately deliver ultra-flexible devices in deep tissue with minimal lesion by the in situ formation of the flexible functional structures only after the surgery process.

Some embodiments of the present disclosure are for an implantation apparatus comprising: (a) a rigid substrate; (b) at least one sacrificial layer; and (c) a flexible implantable device comprised of a first biocompatible polymer layer; a structural layer; a second biocompatible polymer layer. In some embodiments, the at least one sacrificial layer is soluble in aqueous solutions. In some embodiments, the flexible implantable device is configured to become independently movable relative to the substrate as the sacrificial layer dissolves. The substrate in some embodiments is comprised of a semiconducting material, such as, for example, Silicon.

In some embodiments of the present disclosure, the at least one sacrificial layer is configured to dissolve in vivo, in some configurations it is configured to dissolve within 30 minutes in vivo.

In some embodiments of the disclosed devices, part of the structural layer is exposed through the second polymer layer. The structural layer in some embodiments may comprise an electrically conductive component, a fluidic channel, and/or a sensor. In some embodiments, the structural layer and a second polymer layer are configured to allow the attachment of conducting leads to create an electrical path between the structural layer and a circuit external to the implantable device, such as a measurement device, scope, power source, etc.

Some embodiments of the present disclosure comprise a flexible implantable device. Some implantable devices comprise a first biocompatible polymer layer, a structural layer, and a second biocompatible polymer layer. In some embodiments, the implantable device is configured to attach to a rigid substrate by a soluble sacrificial layer such that the implantable device completely detaches from the substrate as the sacrificial layer dissolves.

Some methods of the present disclosure include methods of manufacturing an implantation apparatus. In some embodiments, such method comprises, for example, depositing a sacrificial layer onto a substrate; depositing a first biocompatible polymer layer onto the sacrificial layer; depositing a structural layer onto the first biocompatible polymer where the surface area of the deposited structural layer is less than the surface area of the first biocompatible polymer layer; and depositing a second biocompatible polymer layer onto the structural layer to partially encapsulate the structural layer between the first and second polymer layers.

Some embodiments comprise etching the substrate to a thickness and shape for implantation surgery. Such etching may be done by a variety of means, including Reactive Ion Etching (RIE) and other photolithographic techniques. In some embodiments, the etching process comprises etching part of the substrate from a first perspective through a protection mask window from the side of the substrate on which the first polymer layer and the structural layer were deposited. In some embodiments, this facilitates carving the profile of a probe where the etching depth is close to the desired final thickness of the probe for surgical operation; the shape of the protection mask defines the final shape of the probe; and the protection mask layer is configured to be completely removed after etching. Etching the substrate from a second perspective may continue until the probe is fully suspended.

In some embodiments, the sacrificial layer is comprised of magnesium, aluminum, or a combination of the two metals. In some embodiments, these and/or other bio-soluble materials may be used for the sacrificial layer.

In some embodiments, the etching step compromises preparing a protection mask layer before etching by spin-coating a light-sensitive photoresist on the substrate, exposing the coated substrate to a pattern of ultraviolet light, and chemically removing the exposed area of photoresist.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the term “substantially” may be substituted with “within [a percentage] of” what is specified, where the percentage includes 0.1, 1, 5, and 10 percent.

Further, a device or system that is configured in a certain way is configured in at least that way, but it can also be configured in other ways than those specifically described.

The terms “comprise” (and any form of comprise, such as “comprises” and “comprising”), “have” (and any form of have, such as “has” and “having”), and “include” (and any form of include, such as “includes” and “including”) are open-ended linking verbs. As a result, an apparatus that “comprises,” “has,” or “includes” one or more elements possesses those one or more elements, but is not limited to possessing only those elements. Likewise, a method that “comprises,” “has,” or “includes” one or more steps possesses those one or more steps, but is not limited to possessing only those one or more steps.

Any embodiment of any of the apparatuses, systems, and methods can consist of or consist essentially of—rather than comprise/include/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Some details associated with the embodiments described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the embodiment(s) depicted in the figures.

FIG. 1A is a perspective view of an embodiment of the present thin-film devices after deposition of a sacrificial layer and device layers.

FIG. 1B is a perspective view of an embodiment of the present thin-film devices masked to perform RIE to define the thin-film device and supporting structures.

FIG. 1C is a perspective view of an embodiment of the present thin-film devices after performing RIE from the backside of the device wafer to remove excess materials from the device and supporting structures.

FIG. 1D is a cross-sectional view of the thin film device shown in FIGS. 1A-1C, including the supporting rigid substrate, sacrificial layer, a first passivation layer, a conducting layer, and a second passivation layer.

FIG. 1E illustrates the device probe with a dotted line to indicate the source of the cross-sectional view of FIG. 1D.

FIG. 2 is a bar graph illustrating the biocompatibility of certain sacrificial layer materials, e.g., Magnesium (Mg).

FIG. 3A is a perspective view of an embodiment of the present thin-film devices prior to implantation.

FIG. 3B is a magnified perspective view of a portion of the device shown in FIG. 3A, showing the geometry of the tip of the device where only a portion of the thin film was deposited onto a sacrificial layer.

FIG. 3C is a perspective view of the embodiment in FIGS. 3A and 3B after implantation and dissolution of the sacrificial layer, resulting in a new geometry in the device tip deposited onto the sacrificial layer.

FIG. 3D is a magnified perspective view of a device portion from FIG. 3C after implantation and dissolution of the sacrificial layer, illustrating the 3 dimensional structure of the device relative to its former geometry as shown, for example, in FIG. 3B.

FIG. 4A is a top-down perspective view of an array of thin-film devices in accordance with some embodiments of the present disclosure.

FIG. 4B and FIG. 4C are magnified view of one device in the array shown in FIG. 4A illustrating device structure of a “bend-up” embodiment of the present disclosure.

FIG. 4D is a chart illustrating the relationship between final device geometry after dissolution of the sacrificial layer in certain “bend-up” embodiments of the present disclosure relative to the dimensions (length and width) of conducting layers within the device.

FIG. 5 is a top-down perspective view of two thin-film devices in a single substrate after etching both the top and back of the substrate to define the thin-film device and supporting structure.

FIG. 6 is a magnified view of a portion of the device from FIG. 5 illustrating the tapered tip of the device depicted in FIG. 5.

FIG. 7 is an alternative depiction of the device tip shown in FIG. 6 proximate a neural cell.

FIG. 8 is an illustration of multiple bend-up structures that keep planar structure before implantation defined along the length of an exemplary probe embodiment.

FIG. 9A is a perspective view of one embodiment of the present disclosure prior to implantation of a thin-film device that is attached to substrate substantially or entirely via one or more sacrificial layers.

FIG. 9B is a perspective view of the embodiment shown in FIG. 9A after dissolution of the sacrificial layer, facilitating removal of the supporting substrate.

FIG. 10A-FIG. 10D illustrate one embodiment of the present disclosure through the process of implantation into an agarose gel in which a sacrificial layer dissolves to allow removal of the substrate used for device implantation.

FIG. 10E is a chart illustrating the consistency of device impedance through the implantation process, indicating the stability of the device geometry through the disclosed implantation procedure.

FIG. 11 is a top-down perspective view of the probe tip of a device during fabrication according to some embodiments of the present disclosure.

FIG. 12A is a cross-sectional view of a thin-film device of the present disclosure illustrating one exemplary structure for creating a “bend-up” structured probe where only a portion of the thin-film device is attached to substrate by a sacrificial layer such that the device tip geometry is altered by dissolution of the sacrificial layer.

FIG. 12B is a top-down perspective view of the probe in FIG. 12A after deposition of a first passivation layer, conducting layer, and second passivation layer on top of the sacrificial layer shown in FIG. 12A.

FIG. 13A is a cross-sectional view of the device shown in FIGS. 12A and 12B during top-down etching.

FIG. 13B is a top-down perspective view of the device shown in FIG. 13A after the top-down etching of FIG. 13A.

FIG. 14A is a cross-sectional view of the device shown in FIG. 13B during backside etching.

FIG. 14B is a top-down perspective view of the device of FIGS. 11 through 14A after etching is complete and the device with a partial sacrificial layer may be detached from the supporting substrate structure.

FIG. 15A is a cross-sectional view of an embodiment of the present disclosure where a sacrificial layer forms a base layer for the thin-film device.

FIG. 15B is a magnified top-down perspective view of the device in FIG. 15A, illustrating that the sacrificial layer can extend past the device layers to isolate the thin-film device from the substrate.

FIG. 16A is a cross-sectional view of the embodiment shown in FIG. 15 adding a conducting layer and a second passivation layer to the structure of the device in FIG. 15B.

FIG. 16B is a magnified top-down perspective view of the device in FIGS. 15-16A showing one embodiment for construction of the thin-film device tip, exposing a portion of the conducting layer by limiting the dimensions of the second passivation layer.

FIG. 17A is a cross-sectional view of the embodiment shown in FIGS. 15-16 illustrating the device layers, sacrificial layer, and micro-manipulator substrate layer during top-down etching.

FIG. 17B is a top-down perspective view of the embodiment of FIGS. 15-17A after top-down etching.

FIG. 18A is a cross-sectional view of the embodiment shown in FIGS. 15-17 illustrating the device layers, sacrificial layer, and micro-manipulator substrate layer during back-side etching to remove excess substrate and define the device structure.

FIG. 18B is a top-down perspective view of the device of FIGS. 15 through 18A after etching is complete and the device with a sacrificial layer supporting substantially all of the thin-film device may be detached from the supporting substrate structure.

### DETAILED DESCRIPTION OF THE INVENTION

Various features and advantageous details are explained more fully with reference to the non-limiting embodiments that are illustrated in the accompanying drawings and detailed in the following description. It should be understood, however, that the detailed description and the specific examples, while indicating embodiments of the invention, are given by way of illustration only, and not by way of limitation. Various substitutions, modifications, additions, and/or rearrangements will become apparent to those of ordinary skill in the art from this disclosure.

In the following description, numerous specific details are provided to provide a thorough understanding of the disclosed embodiments. One of ordinary skill in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

### 1. Fabrication and Geometry of Implantable Thin-Film Probes

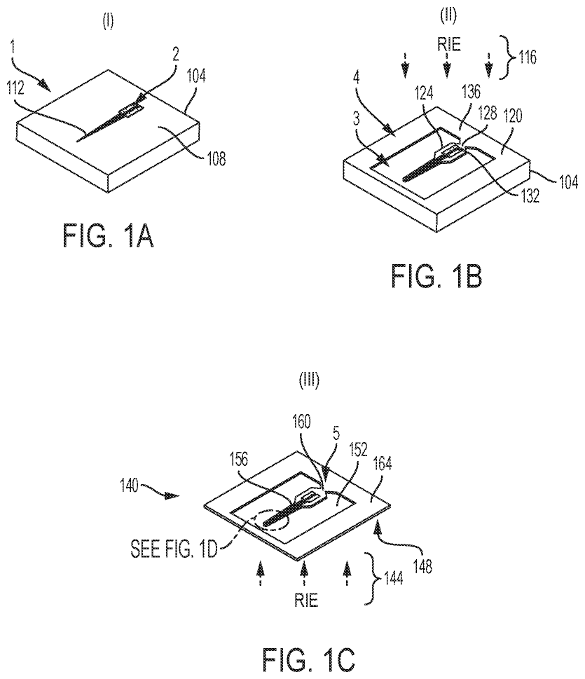

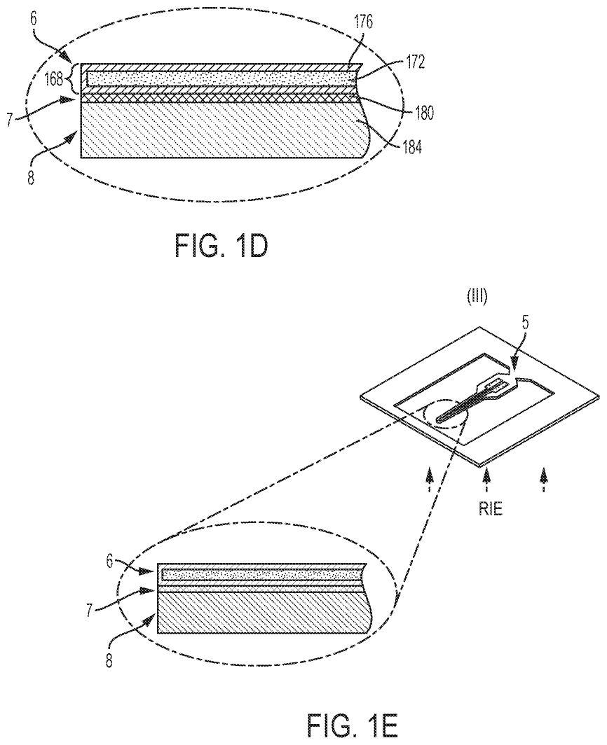

Turning to FIGS. 1A-1D, the schematics of the fabrication procedures according to one embodiment are shown in FIGS. 1A-1C, with the associated probe structure shown in FIG. 1D in a cross sectional view. Specifically, there are three main fabrication stages. In stage (I) shown in FIG. 1A, a double side polished (100) silicon substrate 104 with 150 nm Si₃N₄ coating 108 is first patterned with a biodegradable metal sacrificial layer 112 (discussed in more details below), before a thin-film device structure is fabricated on the top using standard top-down lithographic procedures. The device layer may feature bonding areas for external connections, and a long arm of device(s) that fits into the final shape and dimensions of the probe.

In stage (II), shown in FIG. 1B, a RIE process 116 from the top side is used with a photoresist pattern 120 as the mask to carve out the probe body 124, leaving a thin neck connection 128 between the bonding area 132 and the outer frame area 136. The depth of the RIE in this step defines the final thickness of the probe, typically in the range of 10-30 μm.

In stage (III), shown in FIG. 1C, the whole probe 140 is shaped by a backside RIE process 144, which uniformly reduces the thickness of the whole substrate from the back 148 until the area that has been thinned 152 in the previous stage is completely removed and the probe body 156 is isolated in full suspension. The resulting probe will have a thin neck 160 connection to the supporting frame 164 and can be easily detached by a notch. Note that in this embodiment, three key layers are all defined by lithography as shown in FIG. 1D by way of example, including (1) the top device layer 168 which typically has conductive metal connections 172 passivated by insulating polymer shells 176, (2) the middle biodegradable sacrificial layer 180, and (3) the 10-30 μm thick rigid silicon beam 184 which is key for the precise insertion surgery. In some embodiments, the metal electrode pairs are passivated by SU-8 polymer, but other passivation materials may be used. FIG. 1E illustrates the relationship between the cross-sectional view of FIG. 1D and the probe body in one embodiment.

In some embodiments, a 300 μm thick double-side polished (100) silicon wafer is coated with 150 nm of Si₃N₄ (1), the biodegradable sacrificial layer and thin-film device layer with bonding areas (2) may be fabricated successively using standard top-down lithographic procedures. In some embodiments (e.g., stage II), the probe body is carved out by a top-side RIE process. During the RIE, a photoresist pattern may be used as the etching mask to define and protect the probe profile as well as the outer frame area (4) while the opened area (3) is thinned down to 10-30 μm. In some embodiments (e.g., stage III), the silicon substrate is uniformly etched by a back-side RIE process until the thinned open area (3 from stage II) is completely removed, resulting in a fully isolated probe structure that has a narrow neck connection (5) to the outer supporting frame. In some embodiments, the thin-film device layer (6) and biodegradable metal sacrificial layer (7) are on top of a 10-30 μm thick silicon shaft (8).

In one embodiment, silicon was chosen to construct the initial supporting structure because it can be easily shaped for optimal surgical insertion by standard lithographic processes, and has a Young's modulus of ˜165 GPa so that the critical dimension of the probe can be shrunk down to 10 μm level and still provide enough rigidity for implantation. In some embodiments, the biodegradable material for the sacrificial layer is chosen based on several criteria: (1) compatibility with top-down lithography procedures so that the functional devices can be miniaturized and accurately aligned in a unified fabrication protocol for scalable production; (2) dissolution of the material must only involve the physiological biofluids without the assistance from additional chemicals or enzymes; (3) outcome of the dissolution of the material must not affect the physiological status and functionality of the neighboring cells. In some embodiments, the gradual dissolution of the sacrificial layer in physiological environment triggers the formation, separation, and release of the flexible and functional devices from the rigid supporting beam. In some embodiments, the formation, separation, and release all occur in situ. In some embodiments, the formation, separation, and release occur within a reasonable time frame (15-30 minutes). In some embodiments, the time required for formation, separation, and release facilitates precise implantation such that fine adjustment of the probe position can be allowed after insertion, while the reasonably short time for release allows surgery to be finished without too much waiting or complication. In some embodiments, a thin film device may be stably released into an accurate position during surgery, including films as thin as 1-2 μm. In general, some embodiments of the whole probe, and the fabrication procedures can be used for preparing thin-film probes of varying sizes and shapes for accurate implantation. Some embodiments also facilitate surgical procedures for either a single probe or for an array of probes.

The fabrication of two probe embodiments is now discussed with reference to FIGS. 11-14 and FIGS. 15-18, respectively, as illustrative embodiments to the present disclosure. The first probe embodiment, shown in FIGS. 11-14 may be fabricated with the construction of electrodes 1104 and 1108 and sacrificial layer 1112 on top side 1116 of a 300 μm thick silicon substrate 1120 with silicon nitride coating (150 nm Si₃N₄/100 nm thermal SiO₂ on 300 μm n-Si, double side polished, University Wafers) by standard lithography techniques. Specifically, metal connections 1124 and 1128 and bonding areas 1132 and 1136 (Au/Cr, 55/2 nm) may be patterned on the silicon substrate by standard photolithography, metallization and lift-off processes. The sacrificial layer 1112 pattern may be lithographically defined where the 3D electrodes would be located. A 100 nm Mg layer may be deposited by thermal evaporator (e.g., Cressington 308R) and lift-off by Remover PG (e.g., MicroChem).

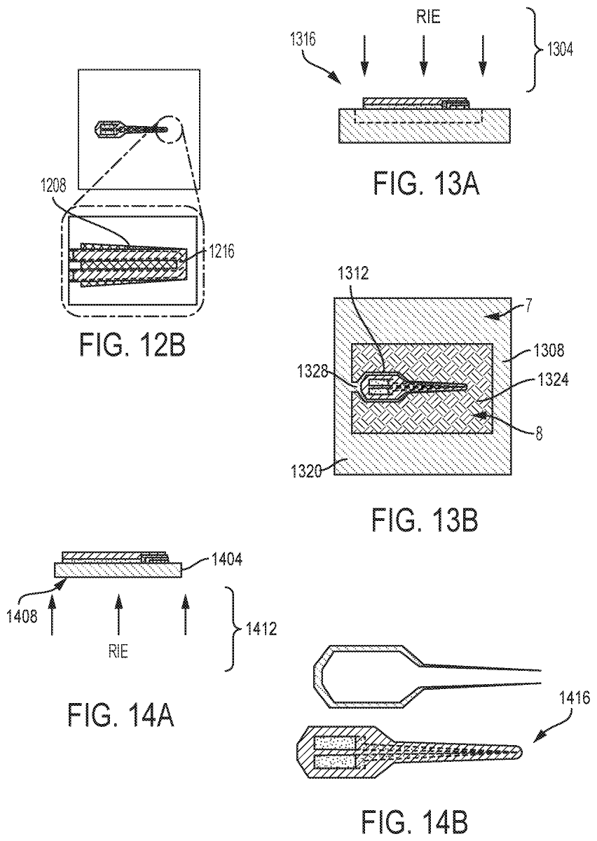

To construct the 3D electrodes, as illustrated, for example, in FIG. 12A, a first layer of 0.5 μm thick SU-8 polymer 1204 (SU-8 2000.5, MicroChem) was shaped by photolithography on top of the sacrificial layer 1208, serving as the bottom passivation layer of the 3D electrodes. Next, electrode pairs with internal stress 1212 (Cr/Pd/Cr, 1.5/75/50 nm) were fabricated on the bottom passivation layer, followed by a second layer of 0.5 μm SU-8 polymer 1216 as the top passivation. FIG. 12B illustrates an alternative view of the cross-section illustrated in FIG. 12A. The final shape of the rigid probe structure for implantation may be defined in the end by RIE (e.g., 1304), as shown in FIG. 13. Specifically, a 3 μm thick photoresist mask 1308 (S1818, MicroChem) was patterned on top side of the silicon substrate by photolithography which defined the shape of a probe 1312. The top surface 1316 (shown in FIG. 13A) of the silicon substrate was then etched by RIE (STS ICP Advanced Silicon Etching, Fluorine) for 10-30 μm. During the etching process, the photoresist mask 1308 protected the probe structure 1312 as well as the supporting frame 1320 while only the open areas 1324 were etched, forming a 10-30 μm thick plateau of the shape of the final probe with one thin neck connection 1328 to the supporting frame 1320.

In some embodiments, as shown in FIGS. 14A and 14B, the silicon substrate 1404 may be etched uniformly from the back side 1408 by an RIE process 1412 until the open windows (e.g., 1324 in FIG. 13B) on the front side (e.g., 1316 of FIG. 13A) are completely removed, resulting in a 10-30 μm thick probe 1416.

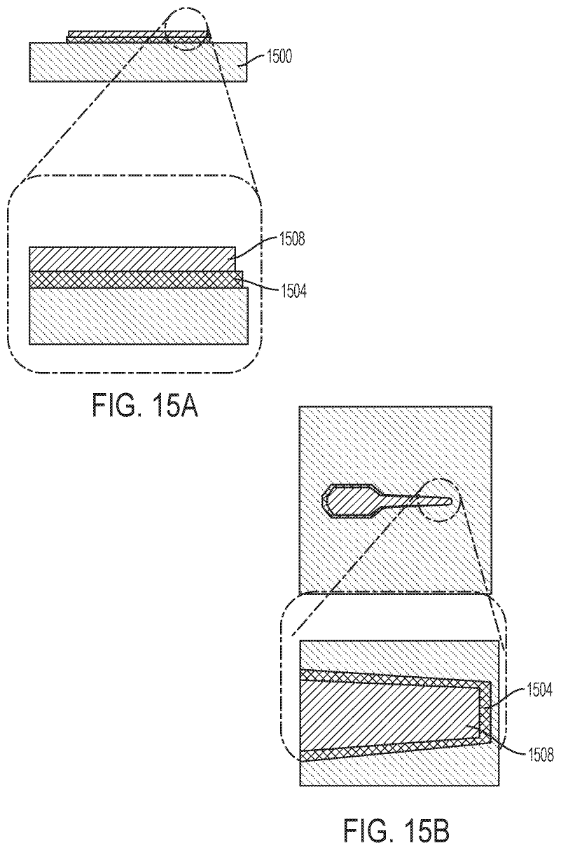

A second probe embodiment, shown in FIGS. 15-18 may be fabricated on a Si₃N₄ coated silicon substrate 1500 (150 nm Si₃N₄/100 nm thermal SiO₂ on 300 μm n-Si, double side polished, University Wafers). In some embodiments, a sacrificial layer 1504 (100 nm Mg by thermal evaporation) was first patterned on the top side of the silicon substrate 1500 by standard lithography techniques, as shown in FIGS. 15A-15B. To construct the fully flexible probe structure, a 5 μm thick SU-8 polymer layer 1508 (SU-8 2005, MicroChem) of the same shape of the final probe was lithography defined on top of the sacrificial layer 1504, serving as bottom passivation layer 1504. This layer 1508 of SU-8 may be thinned down to 3 μm using, for example, oxygen plasma (Plasma-Therm RIE 790), in some embodiments.

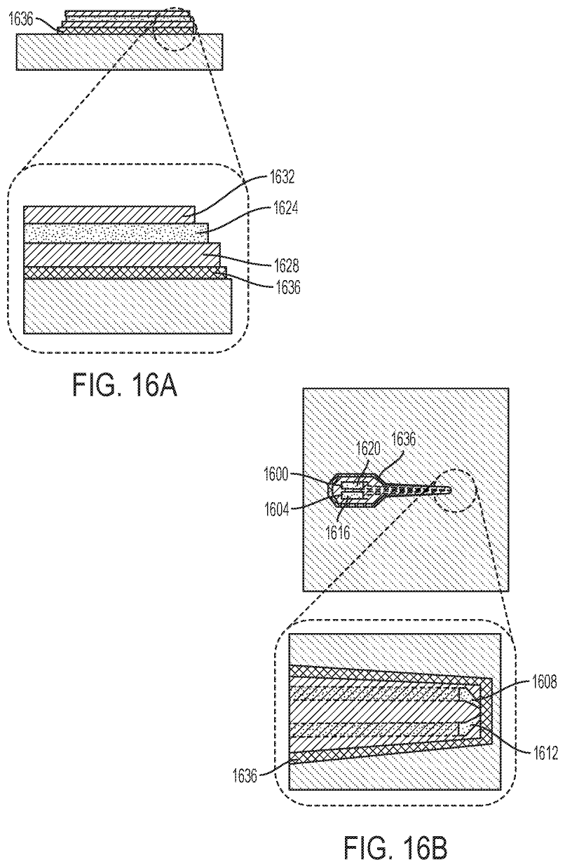

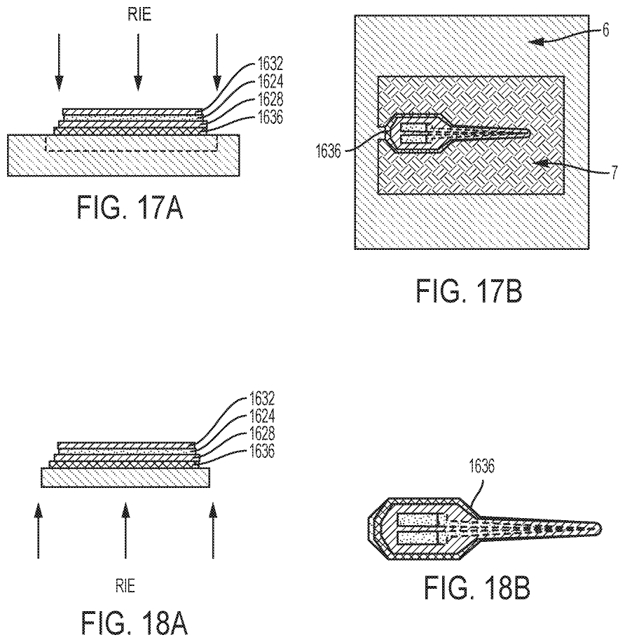

Turning to FIGS. 16A and 16B, electrode pairs 1608 and 1612, metal connections 1616 and 1620, and bonding pads 1600 and 1604 (Au/Cr (e.g., 1624), 50 nm/2 nm) may be fabricated on top of the SU-8 layer 1628, as shown in FIG. 16A-16B, followed by a second 0.5 μm SU-8 polymer layer 1632 (SU-8 2000.5, MicroChem) as the top passivation layer. The etching processes that defined final thickness and shape of the rigid probe structure may follow procedures described above in the first probe embodiment described in reference to FIGS. 11-14, except that the resulting probe has the whole thin-film device structure on top of a continuous layer of sacrificial layer 1636, as shown in FIGS. 17-18.

### 2. Surgical Techniques and Embodiments In Vivo

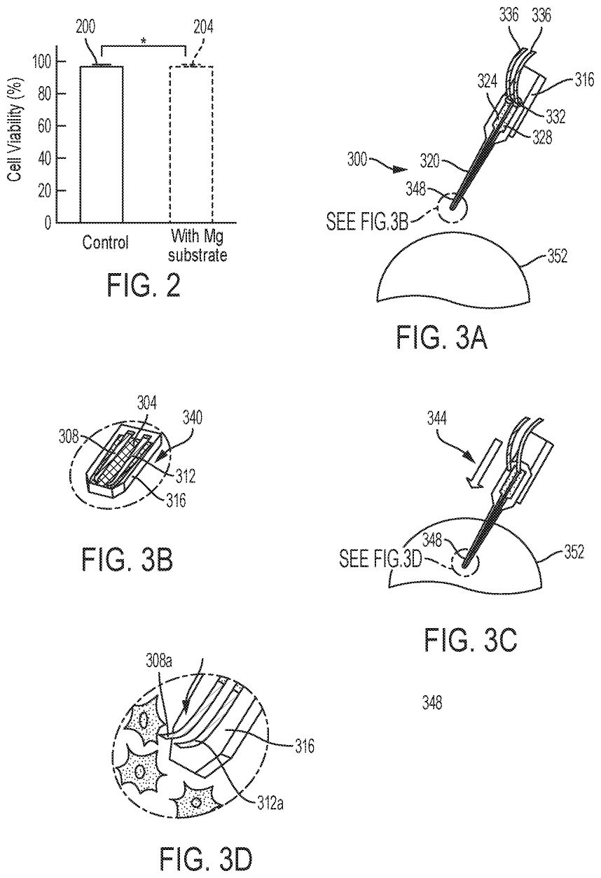

There have been many reports on integrating biodegradable materials into implantable or surface-mounting electronic devices. Most widely studied materials used for packaging or gluing components are organic based, such as poly glycolic acid, poly L-lactic acid, and silk fibroin. To date these materials are either not compatible with top-down lithography, or in the case of photocrosslinkable silk protein, additional enzyme protease XIV is required to initiate a very slow degradation which is not practical for use with live cells in vivo. In addition, carboxymethyl cellulose (CMC) or polyethylene glycol (PEG) based dissolvable needles of hundreds of μm in diameter have been proposed to deliver thin film devices into tissue, but the size of the lesion is overall very large due to the weak mechanical strength and the expansion of probe volume after insertion during the dissolution process could potentially cause secondary damages. On the other hand, inorganic biodegradable materials, including metals, semiconductor and dielectric materials have been investigated for use as electrodes, connections, or surface coatings, but no study of using them as sacrificial layer has been reported. Some embodiments of the present disclosure rely on Magnesium (Mg) to construct a biodegradable sacrificial layer, because it has desirable dissolution rates of 4.8±2.2 μm/h in simulated body fluids, and Mg poses a negligible toxicity risk to live cells when fully digested within a short time. The performance of Mg-based sacrificial layers in terms of dissolution time, shelf life, and biocompatibility have been tested and cell viability is shown in FIG. 2 with control bar graph 200 and tested Magnesium substrate bar graph 204.

Test results of the shelf-life and dissolution of a Mg-based sacrificial layer, starting with a 100 nm Mg layer thermally evaporated on a blank silicon substrate showed strong stability. For example, a freshly prepared sample was stored in an ambient environment (humidity <10%) for 10 weeks and the metal film did not show quality degradation. After 10 week's storage, the Mg layer can be dissolved in 1×PBS within 2 minutes. Cell viability tests also show Mg-based sacrificial layers are well-suited for in vivo systems. In some embodiments a 100 nm Mg layer deposited on a 15×15 mm silicon substrate of which the edges were covered by 10 μm thick SU-8 layer that serves as a height spacer mimicking the typical distance from a cell to the sacrificial layer on probe, cell viability may be maintained. Experimental results of this silicon substrate using a coverslip on which the 7 DIV rat cortical neurons were cultured resulted in substantial viability over 20 hour test. FIG. 2 illustrates expected viability for cells exposed to a Mg-based sacrificial layer based on experimentation. Specifically, cell viability of control group and cells cultured with Mg-evaporated substrates. The viability of cells cultured on Mg-evaporated substrate was 97.7±0.3%. Control group viability is 97.3±0.6%, n=9, total number of cells analyzed=1280, *p=0.22.

Specifically, some embodiments involve dissolution in 1× phosphate buffer solution (PBS) of a 100 nm Mg layer deposited on a blank substrate. Such embodiments have been tested after 10 weeks' storage in ambient environment (relative humidity controlled below 10%). And, the Mg layer dissolved cleanly within 2 minutes.

In some embodiments, a top layer of thin-film devices is fabricated atop the sacrificial layer, the dissolution is limited to progress only from the sidewalls of the sacrificial layer. Therefore, the full disappearance of the sacrificial layer and full release of the device structure with mm size dimensions may be slowed down to 15-30 minutes based on the geometry of some embodiments. In some embodiments, probe shelf life is at least six months.

In some embodiments, the local Mg²⁺ concentration increase is estimated as follows: For simplicity, a 1D model is used where Mg is dissolved from an infinite plane and start diffusion in one direction. Given the diffusion coefficient of Mg²⁺ in solution as 7.05×10⁻⁶ cm²/sec, within 10 minutes the diffusion distance of Mg²⁺ in free medium will be ˜950 μm. Since inside the tissue the diffusion would be constrained by the surrounding cells, we can assume an order of magnitude smaller diffusion distance of ˜100 μm which gives us the higher limit of the impact. For a Mg film of 100 nm, the average local concentration increase is about 0.04 mM. Since the extracellular Mg²⁺ concentration ranges from 2 mM for the brain to 7 mM for the heart, such concentration increase is less than 2% down to 0.6%. Since in real scenarios the diffusion would be a 3D model, this defines the higher limit of concentration change. In addition, the increase of extracellular Mg²⁺ concentration generally will introduce suppression of neuron activities, or cardioprotective effects, therefore the dissolution of Mg sacrificial layer would have minimal influence of the physiological status of live cells, and show no long-term impact on the cells.

Based on the proposed fabrication procedures, two embodiments are shown but others are possible and consistent with this disclosure. In a first probe embodiment, a 3D bend-up structure is spontaneously formed on the thin silicon supporting shaft shortly after the probe is exposed to physiological fluids due to the integrated stress within the thin film structure. In a second probe embodiment, the whole ultra-thin device layer is accurately released with all rigid structure completely removed soon after surgery.

FIG. 3A illustrates the basic structure of a first probe embodiment 300. The Mg sacrificial layer was defined locally (red area 304 in FIG. 3A) below the end terminals 308 and 312 of the thin-film device 300, and the rest of the top layer was directly anchored on the silicon supporting shaft 316. The part of the device arms 308 and 312 on top of the Mg layer 304 may be composed of Cr/Pd/Cr (1.5 nm/75 nm/50 nm). When the Mg layer 304 is dissolved in physiological solution, this free ending of the device will turn from the planar structure into a 3D bend-up structure (e.g., 308a and 312a) shown in FIG. 3B due to the built-in stress. This design facilitates inserting the probe into the tissue for the thin-film device to spontaneously form a 3D structure that deviates away from the lesion caused by the surgery so that only the flexible part of the device will get in contact with the active cells in the surrounding space, as shown generally in FIG. 3B.

The yield of bend-up structure with Mg as sacrificial layer in PBS solutions, and the dependence of bend-up height of the devices on the geometry of the electrodes is consistently high. Specifically, fabrication of different batches of array of locally bend-up structures on a planar silicon substrate, and tested for time and yield of the formation of the structure in PBS solution have shown consistent dissolution time within 20 minutes and yield >93%, independent of the shelf life of the samples (ranges from a couple of weeks up to six months).

FIGS. 3A-3D illustrate a probe with 3D flexible thin-film structure 300. FIGS. 3A-3B depict a probe implantation and FIGS. 3C-3D depicts in situ formation of flexible structure. In FIG. 3A, the backside of silicon shaft 320 is glued to a probe holder 316 of micro-manipulator 316 (brown). The bonding areas 324 and 328 are connected to external instrumentation by silver paste 332 and conductive wires 336. FIG. 3B (magnified view from FIG. 3A) of flexible structures at probe tip 340 showing the thin-film flexible structure (e.g., 308 and 312) is held by Mg biodegradable sacrificial layer 316 before implantation. In FIG. 3B, in situ formation of thin-film flexible structure (e.g., 308a and 312a from FIG. 3D) is shown. The black arrow 344 indicates the insertion direction. FIG. 3D (magnified view from FIG. 3C) of thin-film structure 300 shows, after the probe 348 insertion into tissue 352, Mg sacrificial layer (e.g., 304 in FIG. 3B) typically dissolves in 30 minutes in bio-fluids (sacrificial layer 304 from FIG. 3B no longer shown). The thin-film device 300 fabricated on the sacrificial layer 304 will bend-up, deviating from the surface of the probe 316 to form a 3D structure due to internal stress.

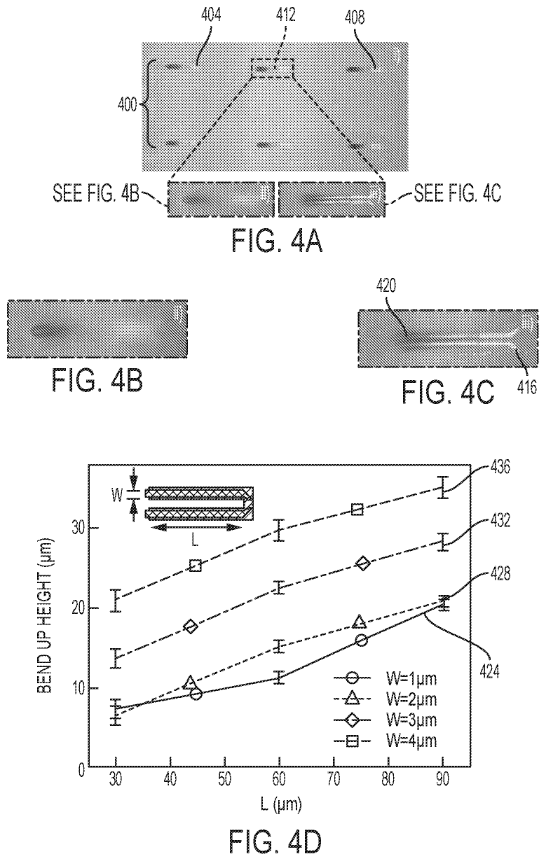

FIG. 4A depicts one embodiment for an array 400 of bend-up electrodes (e.g., 404, 408, and 412) in accordance with the present disclosure. FIGS. 4B and 4C depict a close-up view of a bend-up thin-film electrode structures of an individual probe 412 in array 400. Some embodiments comprise an electrode tip 420 and an anchor part 416 of a same electrode structure, respectively. FIG. 4D shows bend-up height of the thin-film structure as a function of electrode dimensions, where curve 424 represents the bend up height as a function of length for an electrode width of 1 μm. Curves 428, 432, and 436 represent the bend up height as a function of length for electrodes with widths of 2 μm, 3 μm, and 4 μm, respectively. The bend-up height may be positively correlated with electrode length and width. The measurements illustrated in FIG. 4D were done in PBS solution for metal layer with Cr/Pd/Cr thickness of 1.5/75/50 nm. Inset, top-down schematics of electrodes (red) geometry in thin-film structures.

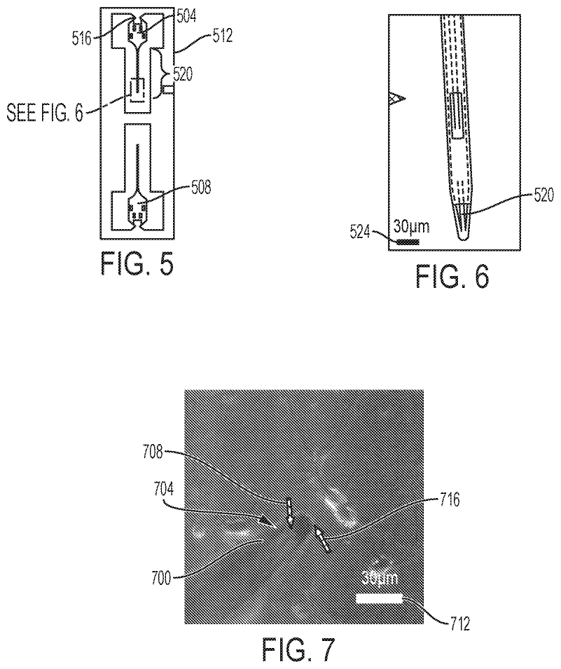

FIG. 5 depicts an embodiment of probes 504 and 508 connected to a supporting frame 512 with a thin neck 516 structure. Some embodiments comprise a tapered probe tip 520 as shown in a magnified view in FIG. 6. The tapered probe tip may be designed to taper within a broad range of size including 1 μm and 30 μm, as indicated by the 30 μm scale bar 524. FIG. 7 illustrates a microscope image of in situ formation of 3D thin-film structure in neuron cell culture medium. Electrodes tip 700 deviated from its original position (indicated by lined arrow 716) to form a 3D structure 704 of which the apex is shown by hatched-patterned arrow 708. Scale bar 712 is shown in this embodiment as 30 μm.

In addition, various embodiments exist with varied width and length of the electrodes of the thin-film devices and the final bend-up height of the structure varies accordingly. In studies where the total width of the electrode pair was kept the same as 8 μm, almost linear increase of bend-up height vs the length of the electrodes may be expected. In embodiments where the width of the electrodes is below 2 μm, the slope of height vs length stayed the same as the 8 μm structures, while when the width increased to 3 and 4 μm, for the same length of the electrodes, the bend-up height increased significantly. This may be explained by the increased stiffness of the electrodes as the ratio of width/length increased. Therefore, for the optimal performance of the probe after implantation where the distance of the final device from the supporting shaft and the flexibility of the functional tips must be controlled, an optimal width of the device electrodes should be chosen.

Some embodiments may include multiple local bend-up structures that keep the planar structure before implantation as shown in FIG. 8 have been defined along the length of the probe body, e.g., bend up probes 804 and 808. Such probes have been tested in vitro to stimulate neurons and may be so used in some embodiments. In some embodiments, the probe is mounted on a micromanipulator with an angle of 25˜30 degrees and the thin-film electrode pairs face down. After inserting the probe into neuron recording medium, the Mg sacrificial layer may be completely dissolved within 15 minutes, and the thin-film electrodes bend downward by ˜20 μm. The end of the electrodes may be aligned with the tip edge of the probe before the bend up. In some embodiments of the device, the device may target and contact individual neurons. In some embodiments, the bend-up of the probe is manipulated such that the access angle of the probe is kept shallow as the rigid tip of the silicon shaft may interfere with the contact when the angle is too steep.

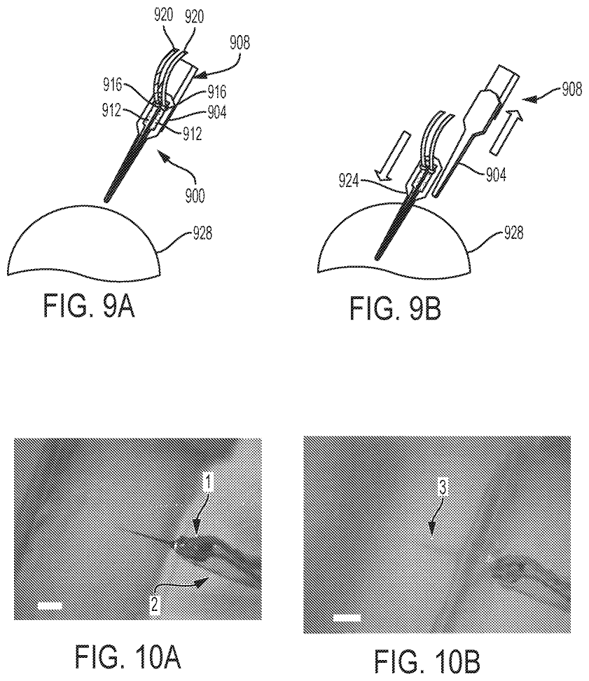

In some embodiments, a fully implanted probe comprises only the flexible structure without any rigid support. Some embodiments will also maintain accurate positioning despite the lack of rigid support. The second probe embodiment may serve this purpose. As shown in FIG. 9A, the second probe type has the whole top thin film structure 900 fabricated on a layer of Mg sacrificial layer covering the rigid silicon shaft 904, shown more clearly in FIG. 9B. Typically, 30 minutes after insertion to the tissue, the whole top film structure 924 is detached from the silicon shaft 904, and the rigid shaft 908 can be retracted following the exact same path of insertion from the tissue, leaving only the thin film staying with its shape and position unaltered. Some embodiments minimize the thickness of the thin-film as well as the rigid structure to be <30 μm. Some embodiments keep the shape and position of the thin-film probe when the thickness was as low as 2 μm, with the rigid shaft structure itself as thin as 10 μm.

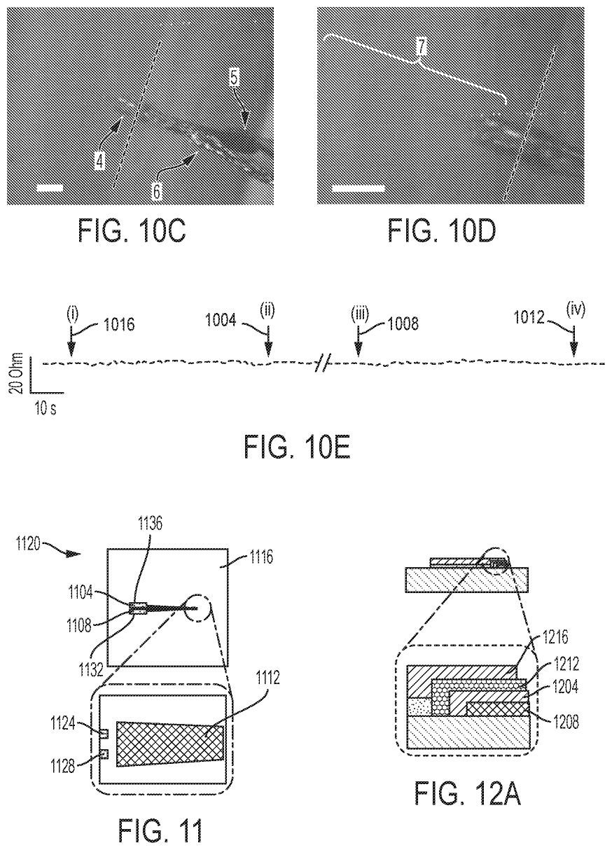

FIGS. 10A-10D show the process of implanting the thin-film probe into a tissue-mimicking agarose gel (1%, Young's modulus 40 kPa). To track the integrity of the film, some embodiments used EBL to connect the two metal electrodes at the very end with a gold thin film of dimensions 2 μm width×2 μm long×50 nm thick so that the resistance through the whole length of the electrodes can be tracked. The calculated resistance based on geometry of the electrodes was ˜1430 ohm, and the measured result before the operation was 1390 ohm. Experimental results of resistance from one embodiment are shown in FIG. 10E. As the probe was inserted into the gel in experiments, it was observed that no distortion of the shape or the direction (FIG. 10B) which showed that the rigidity of the 10 μm thick silicon shaft was enough to deliver the full structure into tissues with similar Young's modulus. The resistance between the electrode pair was constant during this process (FIG. 10E at 1004). After 30 minutes, the silicon shaft was gently pulled back from the gel following the same path, while the thin film probe was left inside the gel (FIGS. 10C and 10E at 1008). As the silicon shaft was fully retracted, the position and shape of the 2 μm thin film probe demonstrated no distortion in shape nor size, and a consistently constant resistance (FIG. 10D and FIG. 10E at 1012). It is important to note that the gold bridge defined by EBL was only protected by a 500 nm SU-8 layer on the bottom and therefore in tests, the thinnest part of the probe was in fact only 550 nm. The fact that the resistance of the probe changed less than 1% throughout the whole process signify the robustness of the disclosed protocol.

Looking to FIGS. 9A and 9B in detail, a fully flexible probe embodiment is shown for implantation. FIG. 9A depicts a silicon backbone 904 of the probe 900 that is glued to probe holder of a micro-manipulator 908. Bonding area 912 on the thin-film device side is connected to external instrumentation by silver paste 916 and conductive wires 920. FIG. 9B depicts the thin-film device layer 924 fully delaminated from the silicon backbone 904 typically in 30 minutes after insertion into tissue 928. The backbone 904 can be fully retracted from the tissue 928 leaving the position and the shape of device 924 intact.

FIGS. 10A-10D are stereomicroscope images show the probe insertion according to one embodiment, tested in a 1% agarose gel mimicking brain tissue environment and detachment of the thin-film device layer. FIG. 10A depicts the bonding pads on thin-film device layer (1) are connected to recording instruments and the silicon backbone is glued to probe holder of micro-manipulator (2). Scale bar is 2 mm. FIG. 10B depicts the probe (3) inserted into tissue-mimicking gel. Scale bar is 2 mm. FIG. 10C depicts the probe 30 minutes after insertion, showing that the sacrificial layer has dissolved. The thin-film device layer (4) is detached from the silicon backbone (5) which is then retracted from the gel, leaving only the thin-film layer and the connected wires (6) in the gel. Scale bar is 2 mm. FIG. 10D depicts an embodiment of the thin-film device layer (7) in the gel. Scale bar is 2 mm. FIG. 10E shows recorded resistance for an experimental embodiment of an electrode pair which are shorted at the tip in the thin-film layer during insertion and thin-film detachment procedures. Time 1016 marks the time when the probe enters gel. Time 1004 represents the time when the probe is completely inserted in the gel. Times 1008 and 1012 mark the beginning of pulling silicon backbone and the fully extraction of silicon backbone, respectively.

Generally, this disclosure also illuminates fabrication frameworks and surgery procedures to prepare ultra-flexible thin-film devices that can be delivered accurately into tissue with biodegradable sacrificial layer which allow (1) in situ formation of 3D bend-up structures after the surgery, and (2) full release of the whole thin-film probe within the tissue and the whole rigid structure completely removed afterwards. The behavior of the bend-up structure with Mg sacrificial layers in biological fluids is disclosed where film probes as thin as unprecedented 2 μm can be delivered accurately with intact geometry inside tissue and the lesion caused by the retractable rigid delivery structure can be as thin as 10 μm using some embodiments of the disclosed devices and techniques.

The above specification and examples provide a complete description of the structure and use of an exemplary embodiment. Although certain embodiments have been described above with a certain degree of particularity, or with reference to one or more individual embodiments, those skilled in the art could make numerous alterations to the disclosed embodiments without departing from the scope of this invention. As such, the illustrative embodiment of the present devices and techniques is not intended to be limited to the particular forms disclosed. Rather, they include all modifications and alternatives falling within the scope of the claims, and embodiments other than the one shown may include some or all of the features of the depicted embodiment. For example, components may be combined as a unitary structure and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not to be interpreted as including means-plus- or step-plus-function limitations, unless such a limitation is explicitly recited in a given claim using the phrase(s) “means for” or “step for,” respectively.

### REFERENCES

1. Sun, Y.; Choi, W. M.; Jiang, H.; Huang, Y. Y.; Rogers, J. A. (Nat. Nanotechnol. 2006, 3, 201-207).
2. Liu, J.; Fu, T.; Cheng, Z.; Hong, G.; Zhou, T.; Jin, L.; Duvvuri, M.; Jiang, Z.; Kruskal, P.; Xie, C.; Suo, Z.; Fang, Y.; Lieber, C. M. (Nat. Nanotechnol. 2015, 7, 629-637).
3. Hong, G.; Fu, T.; Zhou, T.; Schuhmann, T. G.; Huang, J.; Lieber, C. M. (Nano Lett. 2015, 10, 6979-6984).
4. Hwang, S.; Tao, H.; Kim, D.; Cheng, H.; Song, J.; Rill, E.; Brenckle, M. A.; Panilaitis, B.; Won, S. M.; Kim, Y.; Song, Y. M.; Yu, K. J.; Ameen, A.; Li, R.; Su, Y.; Yang, M.; Kaplan, D. L.; Zakin, M. R.; Slepian, M. J.; Huang, Y.; Omenetto, F. G.; Rogers, J. A. (Science 2012, 6102, 1640-1644).
5. Kim, T.; McCall, J. G.; Jung, Y. H.; Huang, X.; Siuda, E. R.; Li, Y.; Song, J.; Song, Y. M.; Pao, H. A.; Kim, R.; Lu, C.; Lee, S. D.; Song, I.; Shin, G.; Al-Hasani, R.; Kim, S.; Tan, M. P.; Huang, Y.; Omenetto, F. G.; Rogers, J. A.; Bruchas, M. R. (Science 2013, 6129, 211-216).
6. Koh, A.; Gutbrod, S. R.; Meyers, J. D.; Lu, C.; Webb, R. C.; Shin, G.; Li, Y.; Kang, S.; Huang, Y.; Efimov, I. R.; Rogers, J. A. (Adv. Healthc. Mater. 2016, 3, 373-381).
7. Luan, L.; Wei, X.; Zhao, Z.; Siegel, J. J.; Potnis, O., Tuppen, C. A.; Lin, S.; Kazmi, S.; Fowler, R. A.; Holloway, S.; Dunn, Andrew. K.; Chitwood, R. A.; Xie, C. (Sci. Adv. 2017, 3, 1-9).

1.-7. (canceled)

8. An implantation apparatus comprising:
(a) a rigid substrate;
(b) at least one sacrificial layer; and
(c) a flexible implantable device comprised of:
a first biocompatible polymer layer;
a structural layer;
a second biocompatible polymer layer;
where the at least one sacrificial layer is soluble in aqueous solutions and where the flexible implantable device is configured to become independently movable relative to the substrate as the sacrificial layer dissolves
where the structural layer and second polymer layer are configured to allow the attachment of conducting leads to create an electrical path between the structural layer and a circuit external to the implantable device.

9. (canceled)

10. (canceled)

11. A method of manufacturing an implantation apparatus comprising:
(a) depositing a sacrificial layer onto a substrate;
(b) depositing a first biocompatible polymer layer onto the sacrificial layer;
(c) depositing a structural layer onto the first biocompatible polymer where the surface area of the deposited structural layer is less than the surface area of the first biocompatible polymer layer; and
(d) depositing a second biocompatible polymer layer onto the structural layer to partially encapsulate the structural layer between the first and second polymer layers;
(e) etching the substrate to a thickness and shape for implantation surgery where the etching process comprises:
(i) etching part of the substrate from a first perspective through a protection mask window from the side on which the layers in step (a) through (d) of claim 10 are deposited to carve out the profile of a probe where the etching depth is close to the desired final thickness of the probe for surgical operation; the shape of the protection mask defines the final shape of the probe; and the protection mask layer is configured to be completely removed after etching;
(ii) etching the substrate from a second perspective until the probe is fully suspended.

12. The method of claim 11 where the etching process is reactive ion etching.

13.-14. (canceled)

15. A method for surgical implantation comprising:
(a) attaching conducting leads to the structural layer of a flexibly implantable device to electrically connect the structural layer to an external instrument;
(b) bonding the rigid substrate onto a holder that is able to move in three dimensions;
(c) positioning the holder to implant the rigid substrate in tissue;
(e) retracting the holder to remove the rigid substrate from the tissue after the sacrificial layer dissolves;
wherein the flexible implantable device comprises
(i) a first biocompatible polymer layer;
(ii) a structural layer;
(iii) a second biocompatible polymer layer;
where the implantable device is configured to attached to a rigid substrate by a soluble sacrificial layer such that the implantable device completely detaches from the substrate as the sacrificial layer dissolves.

16. An implantation apparatus of claim 8 where the substrate is semiconductor.

17. An implantation apparatus of claim 8 where the substrate is silicon.

18. The apparatus of claim 8 where the at least one sacrificial layer is configured to dissolve in vivo.

19. The apparatus of claim 8 where the at least one sacrificial layer is configured to dissolve within 30 minutes in vivo.

20. The apparatus of claim 8 where part of the structural layer is exposed through the second polymer layer.

21. The apparatus of claim 8 where the structural layer comprises an electrically conductive component, a fluidic channel, or a sensor.

22. The method of claim 11 where the sacrificial layer is comprised of magnesium, or aluminum, or a combination of the two metals.

23. The method of claim 11 wherein step (e) further comprises preparing a protection mask layer before etching by spin-coating a light-sensitive photoresist on the substrate, exposing the coated substrate to a pattern of ultraviolet light, and chemically removing the exposed area of photoresist.

24. The method of claim 15 where the sacrificial layer is comprised of magnesium, or aluminum, or a combination of the two metals.

25. The method of claim 15 where the substrate is semiconductor.

26. The method of claim 15 where the substrate is silicon.

27. The method of claim 15 where the at least one sacrificial layer is configured to dissolve in vivo.

28. The method of claim 15 where the at least one sacrificial layer is configured to dissolve within 30 minutes in vivo.

29. The method of claim 15 where part of the structural layer is exposed through the second biocompatible polymer layer.

30. The method of claim 15 where the structural layer comprises an electrically conductive component, a fluidic channel, or a sensor.
